(19) United States
(12) Patent Application Publication
(10) Pub. No.: US 2007/0048734 A1
(43) Pub. Date: Mar. 1, 2007

(54) NOVEL ENHANCED PROCESSES FOR DRUG TESTING INCLUDING NEOPLASMIC TISSUE SLICES AND PRODUCTS THEREBY

(75) Inventor: Sung-Soo Park, Rancho Palos Verdes, CA (US)

Correspondence Address: GREENBERG TRAURIG LLP, 2450 COLORADO AVENUE, SUITE 400E, SANTA MONICA, CA, 90404, US

(73) Assignee: HEPAHOPE, INC.

(21) Appl. No.: 11/468,502

(22) Filed: Aug. 30, 2006

Related U.S. Application Data
(60) Provisional application No. 60/712,964, filed on Aug. 30, 2005.
(60) Provisional application No. 60/782,029, filed on Mar. 13, 2006.
(60) Provisional application No. 60/785,308, filed on Mar. 23, 2006.
(60) Provisional application No. 60/791,966, filed on Apr. 14, 2006.

(51) Int. Cl. C12Q 1/00

(52) U.S. Cl. 435/4

(57) ABSTRACT

A novel method for testing neoplasmic tissue in a testing system is more effective than conventional cell culture systems and functions by treating the neoplasmic tissue slice system's samples with at least one compound and observing the effect on the neoplasmic tissue slices resident therein, or cells, tissue samples or other derivatives from the testing process, among other things

### CROSS-REFERENCE TO RELATED APPLICATIONS

This application claims priority from U.S. Provisional Patent Application Ser. Nos. 60/712,964 filed on Aug. 30, 2005, 60/782,029 filed on Mar. 13, 2006, 60/785,308 filed Mar. 23, 2006, and 60/791,966 filed on Apr. 14, 2006. The contents of each of these applications are hereby expressly incorporated by reference, as if fully set forth herein and full Paris Convention Priority is hereby expressly claimed.

Likewise, expressly incorporated by reference herein are U.S. Pat. No. 5,773,285 issued Jun. 30, 1998, U.S. Pat. No. 5,795,710 issued Aug. 15, 1998 and U.S. Pat. No. 5,976,870 issued Nov. 2, 1999. Also incorporated by reference herein are PCT Application Nos. US/2004/015824 (PCT Publication No. WO 2005/000376) and US/2004/16477 (PCT Publication No. WO 2005/061694), and any divisionals, extensions, patents of addition, or National Stage filings of the same.

### BACKGROUND OF THE DISCLOSURE

The invention relates to systems for testing used with biological tissue slices, including those derived from any major organ, organ system, cloned tissue using somatic cell transfer, or any other stem cell based regimen, including cord blood, any manner of neoplasms. The instant tester may be used to evaluate, detect, and test drug candidates, drugs, and drug metabolites as a method of providing personalized medical treatments. Finally, the instant tester can be used to study diseases, such as carcinogenesis, in tissue that has been selected based upon phenotypic analysis, or any other proteomics, genomics, or metabonomic analysis methods, including nano-system biological approaches.

It has been established that current regimens have two major failings with respect to pre-market and post-market testing. By way of example, the VIOXX® debacle made it clear that efforts to screen candidates for specific disease state treatments is needed to find out if segments of the populace have the potential for adverse reactions. Using currently available genomic and proteomic analysis methods, high risk patients and categories of patients could have tissue screened in advance of being subject to such potentially harmful and morbidly toxic compounds.

Likewise, escalating costs impact this calculus and underscore and highlight the needs for the teachings of the present disclosure. This becomes crystal clear upon review of the historical numbers which have been established in this space.

In 2001, the average cost to develop a new drug exceeded $800 million, according to a study by the Tufts Center for the Study of Drug Development. Of this, approximately $16 million on average per company was used for pre-clinical research. Reduction of testing time and cost in drug development is therefore a critical factor to the survival of most pharmaceutical companies. In addition, since there is usually more than one company competing in the same drug arena, any competitive advantage is welcome. A major portion of drug development costs is borne during the FDA approval process. However, much of this cost cannot be managed in the same way that pre-clinical costs can. To address soaring pre-clinical costs, more efficient, affordable, and timely methods of in vivo and in vitro testing and selection of potential new drug candidates are of significant interest in the industry.

In developing a new drug, toxicity is always an important consideration. Since the liver metabolizes most drugs, liver damage is of great concern. Likewise, other organs and systems, and how they react to foreign substances, is extremely important. Conventional in vivo and in vitro tests utilizing small animals and cell culture techniques are therefore widely used to assess liver function in drug development. However, these conventional tests have particular disadvantages, such as individual variation, high costs to use large animals, and loss of naturally existing characteristics of liver in situ. The same is true for other organs. As our knowledge base increases on these other organs and how they bring insight into how mammals respond to various pro-drugs, drugs, compounds and systems, the relative significance of the instant disclosure becomes more prominent and significant.

To overcome these disadvantages, cell culture systems have been used. However, with these models cell-to-cell connective interactions cannot be maintained for a desired length of time. Once cell-to-cell connectivity is lost, failure of the testing scheme soon follows because it is no longer directed to organ, system, or organism level response.

Bioartificial organ devices are currently in development. It is believed that organ function can only be replaced with the biological substrate, that is, for example, liver slices or a whole liver specimen, which requires the availability of liver tissue from xenogenic or neoplasmic sources. Recent efforts have combined mechanical and biologic support systems in hybrid liver support devices. The mechanical component of these hybrid devices serves both to remove toxins and to create a barrier between the patient's serum and the biologic component of the liver support device. The biologic component of these hybrid liver support devices may consist of liver slices, granulated liver, or hepatocytes from low-grade tumor cells or porcine hepatocytes. These biologic components are housed within chambers often referred to as bioreactors. However, problems remain with respect to maintaining the functionality of the individual cell lines used in these devices. Most devices use immortalized cell lines. It has been found that over time these cells lose specific functions.

There are several groups developing bioartificial liver devices, for example, Circe Biomedical® (Lexington, Mass.), Vitagen® (La Jolla, Calif.), Excorp Medical (Oakdale, Minn.), and Algenix (Shoreview, Minn.). The Circe Biomedical device integrates viable liver cells with biocompatible membranes into an extracorporeal, bioartificial liver assist system. Vitagen's ELAD® (Extracorporeal Liver Assist Device) Artificial Liver is a two-chambered hollow-fiber cartridge containing a cultured neoplasmic liver cell line (C₃A). The cartridge contains a semipermeable membrane with a characterized molecular weight cutoff. This membrane allows for physical compartmentalization of the cultured neoplasmic cell line and the patient's ultrafiltrate. Algenix provides a system in which an external liver support system uses porcine liver cells. Individual porcine hepatocytes pass through a membrane to process the neoplasmic blood cells. Excorp Medical's device contains a hollow fiber membrane (with 100 kDa cutoff) bioreactor that separates the patient's blood from approximately 100 grams of primary porcine hepatocytes that have been harvested from purpose-raised, pathogen-free pigs. Blood passes though a cylinder filled with hollow polymer fibers and a suspension containing billions of pig liver cells. The fibers act as a barrier to prevent proteins and cell by-products of the pig cells from directly contacting the patient's blood but allow the necessary contact between the cells so that the toxins in the blood can be removed.

Various aspects of these devices represent improvements over pre-existing technology, but they still have particular disadvantages. The effectiveness of these devices, all of which use individual hepatocytes, is limited due to the lack of cell-to-cell interactions, which characterize the liver in its in vivo state. Accordingly, a bioartificial organ, for example a liver with improved efficiency, viability, and functionality for use in drug development would be beneficial. This longstanding need is addressed by the instant teachings, which provide for drug testing with bio-artificial tissue slices.

As the technology of bioartificial organ systems continues to advance, improved methods screening compounds also develop. Disclosed in this application are methods that utilize the recent improvements to bioartificial organ systems, and specifically apply the developed protocols to neoplasmic tissue.

### SUMMARY OF THE DISCLOSURE

Disclosed is a novel method for testing tissue in a bioartificial organ system, cell culture, and neoplasmic tissue itself, by treating the bioartificial organ system or cell culture with at least one compound and observing the effect on the bioartificial organ system or cell culture. Likewise, those skilled in the art readily understand that further disclosed is a business method for using the apparatus and methods of the present disclosure to provide for tissue and organ specific screening for patients in complement with cutting edge genomic, proteomic, and metabonomic analysis.

Disclosed herein is a method for providing simulated in vivo conditions comprising providing a bioreactor for substantially duplicating in vivo tissue function in vitro, providing for the bioreactor to hold at least one aliquot of a tissue sample, and allowing at least one aliquot to generate useful data.

Likewise, a method for substantially simulating in vivo conditions is disclosed, comprising obtaining a bioreactor for substantially duplicating in vivo tissue function in vitro, obtaining a tissue sample, and using at least one aliquot of the tissue sample to generate useful data.

Still further disclosed is a method for substantially simulating in vivo conditions comprising providing a neoplasmic tissue sample, dividing the tissue sample into tissue slices, including the tissue slices as a part of a bioartificial tissue system, and allowing the bioartificial tissue system to be used by treating the tissue slices with at least one drug regimen to generate useful data.

A similar method is disclosed comprising obtaining a bioartificial tissue system containing neoplasmic tissue slices, using the bioartificial tissue system by treating the tissue slices with at least one drug regimen to generate useful data, and comparing the data to determine mitotic activity, toxicity of a compound, or histopathology, choosing a drug regimen based on the comparisons of data.

Finally, a business method for neoplasmic tissue testing which comprises providing a bioreactor-based system for housing neoplasmic tissue slices, populating the bio-reaction based system with neoplasmic tissue, testing a regimen on the neoplasmic tissue, and collecting results.

### BRIEF DESCRIPTION OF THE FIGURES

The above-mentioned features and objects of the present disclosure will become more apparent with reference to the following description taken in conjunction with the accompanying drawings wherein like reference numerals denote like elements and in which:

FIG. 1 is a schematic diagram of a system of an embodiment of a bioartificial organ system;

FIG. 2 is a perspective view of an embodiment of a neoplasmic tissue and bioartificial organ system, according to the instant disclosure;

FIG. 3 is a perspective view of an embodiment of a bioreactor installed in a neoplasmic tissue and bioartificial organ system, according to the instant disclosure;

FIG. 4 is a perspective view of an embodiment of the system of FIG. 1-FIG. 3;

FIG. 5 is a perspective view of an embodiment of the instant system showing the placement of a neoplasmic tissue slice;

FIG. 6 is an exploded view of an embodiment of a tissue slice apparatus containing a neoplasmic tissue slice;

FIG. 7A is a side sectional view of a tissue slice arrangement of an embodiment of a neoplasmic tissue and bioartificial organ system;

FIG. 7B is a perspective view of the neoplasmic tissue slice arrangement of FIG. 7A;

FIG. 8 is a graphical representation of in vitro lidocaine clearance with continuous and intermittent perfusion using a bioartificial organ system;

FIG. 9 is a graphical representation of in vitro lidocaine clearance with a 6-hour and a 24-hour run using the bioartificial organ system;

FIG. 10 is a graphical representation of in vitro DMX concentration with a 6-hour and a 24-hour run using the bioartificial organ system; and

FIG. 11 is a graphical representation of in vitro ammonia clearance with a 6-hour and a 24-hour run using the bioartificial organ system;

FIG. 12 is a schematic showing the process undertaken by a neoplasmic tissue slice apparatus of the present disclosure;

FIG. 13 is a flowchart of an embodiment demonstrating a method of using a neoplasmic tissue and bioartificial organ system to obtain useful results, according to embodiments of the present disclosure.

### DETAILED DESCRIPTION

As used in the present disclosure, the term “regimen” shall be understood to mean one or more drugs, compounds, therapeutic agents, nucleic acids, peptides, metabolites, viruses, bacteria, or other agents that may be applied to a cell or tissue.

The present inventor has discovered an improved modular system for circulating plasma about slices of organs from animals to create a system for neoplasmic tissue testing, inter alia.

Another object of the present disclosure is to provide an effective method using a platform for testing of the efficacy of compounds prior to actual administration of the compounds to live patients. The compound's toxic and pharmacologic effects are realized through in vivo and in vitro animal testing. However, the present disclosure allows for the use of tissues, both neoplasmic and human, to be cultured and for the testing of the compound on the tissue. For new drug compounds, the FDA will ask, at a minimum, the new drug applicant to: (1) develop a pharmacologic profile of the drug; (2) determine the acute toxicity of the drug in at least two species of animals; and (3) conduct short-term toxicity studies ranging from 2 weeks to 3 months, depending on the proposed duration and use of the substance in the proposed clinical studies. The process is complicated and costly, with hundreds and sometimes thousands of compounds being tested.

A further object of the present disclosure is to provide a method upon which a drug regimen, for example, a chemotherapeutic regimen, can be personalized for individual users. Generally speaking, with chemotherapeutic regimens, not all regimens work the same way in all patients. A drug cocktail that is effective in one patient will be ineffective in a different patient. The present disclosure provides a method to test the efficacy of a drug regimen prior to administration to patients, thereby administering only the most effective regimen to each patient.

A further object of the present disclosure is to provide a research platform and method for the study of disease and the ways in which compounds affect a tissue, particularly using neoplasmic tissue.

For the purposes of this disclosure, the term tissue sample, tissue slice, or tissue aliquot refers to a bioartificial organ system or cell cultures of tissue cells that are not part of a bioartificial organ system, and particularly neoplasmic tissue.

In accordance with an embodiment of the present disclosure, there is provided a neoplasmic tissue testing method that uses a bioartificial organ system for evaluation, detection, and testing of drug candidates, drugs and drug metabolites as incorporated by reference. The system has a neoplasmic tissue slice culture apparatus. Other similar systems that allow for testing of neoplasmic tissue and bioartificial organs are expressly contemplated. Moreover, in other embodiments, the methods of the present disclosure may be used with conventional tissue samples. However, neoplasmic tissue is most effective for getting real time results and realistic outcomes, when combined with human tissue in testing.

The present disclosure provides a method for neoplasmic tissue testing that provides a way to personalize chemotherapeutic regimens in individual patients, to predict toxicity of a compound in normal tissues, and to study disease. According to an embodiment, during surgery, for example a biopsy, a sample of tissue is extracted. The sample is sliced into a plurality of tissue slices. Various chemotherapeutic regimens are applied to each tissue slice. After the regimen is complete, the results are compared to determine the efficacy of each regimen. By comparing various regimens, personalized chemotherapeutic regimens may be designed based on the results from the tests. Other applications for an improved tester, as disclosed are to study and evaluate toxicity, and study and evaluate histopathology.

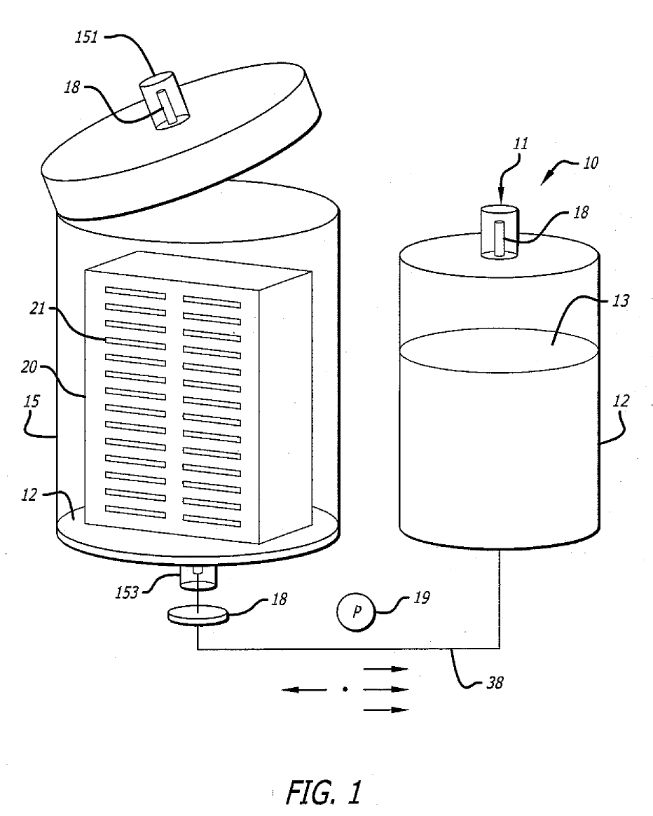

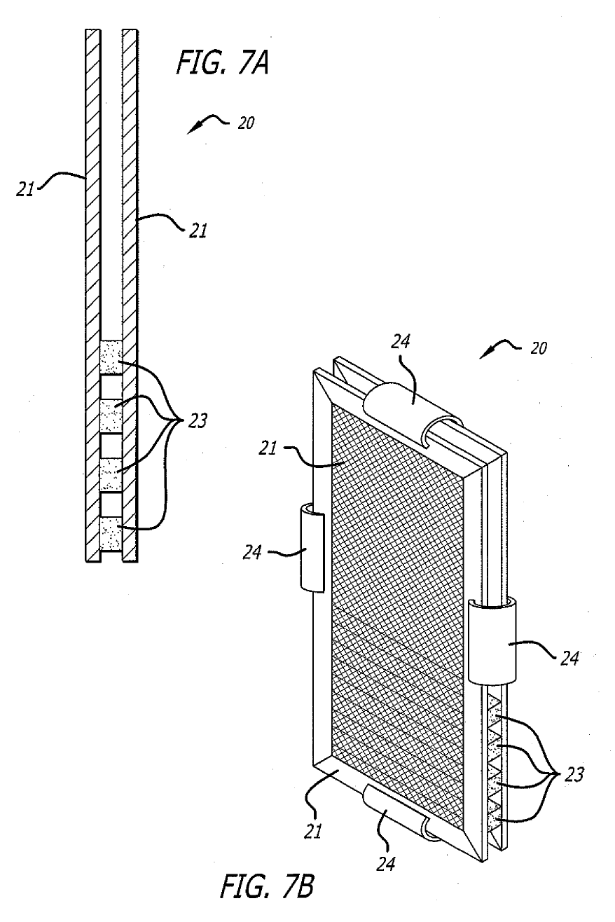

FIG. 1 is a schematic representation of an embodiment of a drug testing system 10 in accordance with the present disclosure. From reservoir 12 culture medium 13 is introduced into the bioreactor 15. Within bioreactor 15 is at least one tissue slice apparatus 20, which comprises at least one tissue slice 23 arranged between two wire meshes 21 (see FIGS. 7A and 7B) and placed vertically parallel within bioreactor 15. As culture medium is introduced into the bioreactor, the culture medium level begins to rise until it comes into contact with the tissue slices, which allows tissue slices 23 to contact a myriad of compounds that are introduced via the culture medium.

Those skilled in the art understand that reservoir 12 is likewise interchangeable with a plurality of related solutions, mechanisms, apparatus and processes. Likewise, machines or tools performing the functions of the same can be freely interchanged with reservoir 12, without departing from the scope of the instant disclosure.

Oxygenated gas is introduced by gas valve 151 in the top of the chamber Although the gas valve is shown in the top of the chamber, it is also contemplated herein that the gas valve could be on the side or bottom of the chamber, provided with an appropriate seal to prevent leakage of liquid medium. The gas is preferably a mixture of 95% O₂ by volume and 5% CO₂ by volume, and is supplied at a pressure ranging from 1 to 10 ATM to the chamber through the gas valve and discharged therefrom, while controlling the pressure by a pressure controller (not shown). A solenoid valve (also not shown) may be coupled with the pressure controller to maintain a pre-set gas pressure. Gas sterilizing device 18, for example, a syringe filter having a pore size of about 0.22 μm, is preferably installed in gas valve 151 to filter out microbes, thereby sterilizing the supply gas to the chamber. Gas check valve 11 with gas sterilizing device 18 is positioned on the medium reservoir and serves to equalize the pressure between the reservoir and atmosphere.

Stabilization of the tissue slices, including neoplasmic tissue is an important feature of the invention. The tissue slices are cultured under the supplies of the culture medium and an oxygenated gas. The liquid culture medium, or the plasma, is supplied through the reservoir into the chamber and the oxygenated gas is supplied through the top of the chamber. Each is supplied at regular intervals so that each of the neoplasmic tissue slices is exposed alternately to the medium and to the gas at an exposure-time ratio ranging from about 1:1 to about 1:4. A ratio of about 1:2.5 to about 1:3.5 has been found to be effective, and a ratio of about 1:1 or 1:3 has also been found to be effective, although changing these parameters are certainly within the normal skill level of an artisan. Pump 19 controls the flow of the culture medium. The rate in which a tissue slice is alternately exposed to gas and culture medium corresponds roughly to the rate of metabolism.

In the present disclosure Waymouth MB 752/1 culture medium is preferred over plasma. The particular choice of the type of culture medium or plasma will be known to a person of ordinary skill and may vary from cell type to cell type. To prevent central necrosis, the gas mixture described above is 95% O₂ and 5% CO₂. Since this mixture may produce free oxygen radicals, which are often toxic to tissue culture cells, care must be taken. For example, with liver samples a high concentration of glutathione and vitamin E, as oxygen free radical scavengers and anti-oxidants, are added and supplemented with 10% inactivated fetal bovine serum and L-glutamine.

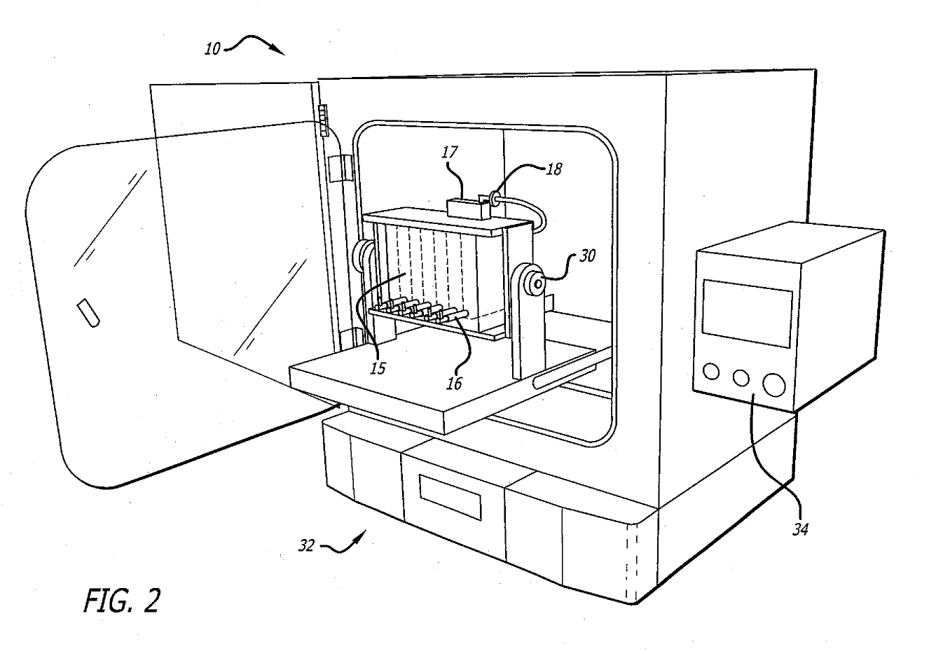

Referring now to FIG. 2, an embodiment of a neoplasmic tissue and bioartificial organ system 10 is shown. Bioartificial organ system 10 comprises one or more bioreactors 15 disposed in incubator 32 that regulates temperature and humidity within the chamber. Incubator 32 allows users to regulate the conditions of a bioartificial organ, ensuring that the bioartificial organ is exposed to optimal conditions for viability over time. The choice of a suitable incubator system for tissue culture is well known in the art and requires no further recitation.

Disposed in an incubation chamber are one or more bioreactors 15. Each bioreactor 15 holds one or more neoplasmic tissue slices or samples. Rotator 30 turns bioreactor 15 for wet and dry phases respectively. The wet phase corresponds to the time that the tissue slice is substantially exposed to culture medium. Likewise, the dry phase corresponds to the time the tissue slice is substantially exposed to gas. Control module 34 provides an interface for controlling the parameters of bioartificial organ system 10 operation. For example, using control modules, the time period that neoplasmic tissue slices are exposed to gas and culture medium may be regulated, which roughly corresponds to metabolism rates. Similarly the gas to culture medium ratios may be regulated using control module 34, as well as other necessary operating parameters. Within incubator 32, gas and culture medium supplies are provided as would be understood by a person of ordinary skill in the art.

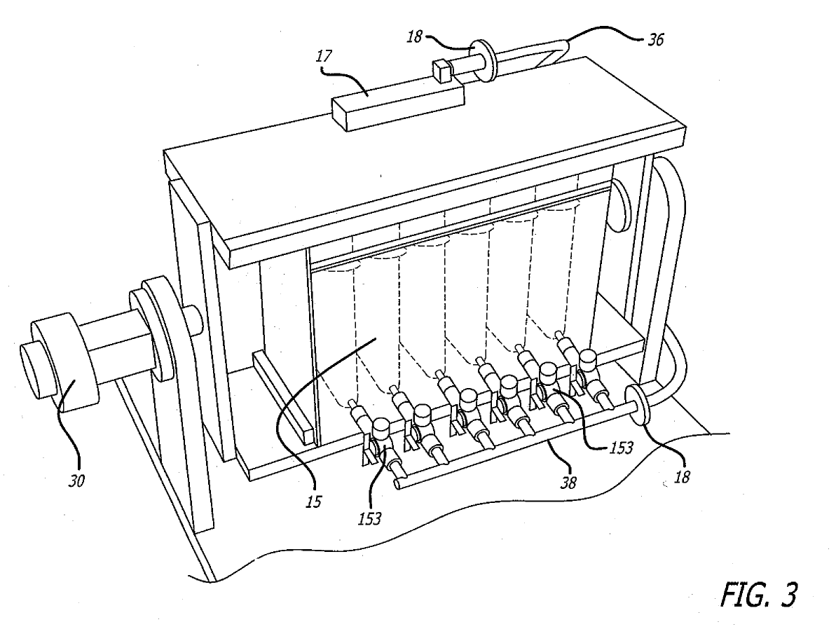

Turning now to FIG. 3, there is shown a close-up view of a bioreactor 15 installed in incubator 32. For ease of ingress and egress, bioreactor 15 and rotator 30 may be affixed to a platform that slides into and out of incubator 32. When installed in incubator 32, each bioreactor 15 is connected to gas and culture medium supplies, 36 and 38 respectively. As shown in the embodiment of FIG. 3, gas valves 151 (one for each tissue slice apparatus chamber 155) connects to gas supply 36. As shown in FIG. 3, manifold system 17 is disposed between gas valves 151 and gas supply 36. Gas filter 18, as previously described, is installed between gas supply 36 and gas valves 151. Similarly, culture medium valves 153 are connected to culture medium supply 38. Culture medium filter 18, as previously described, is disposed between culture medium supply 38 and culture medium valves 153.

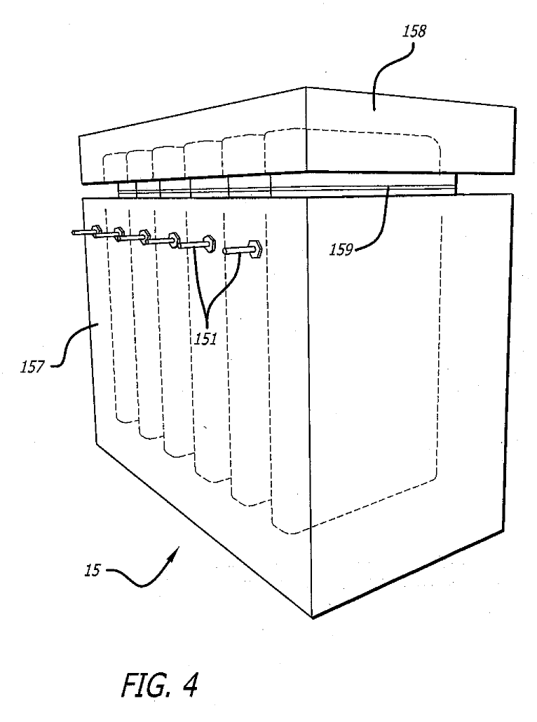

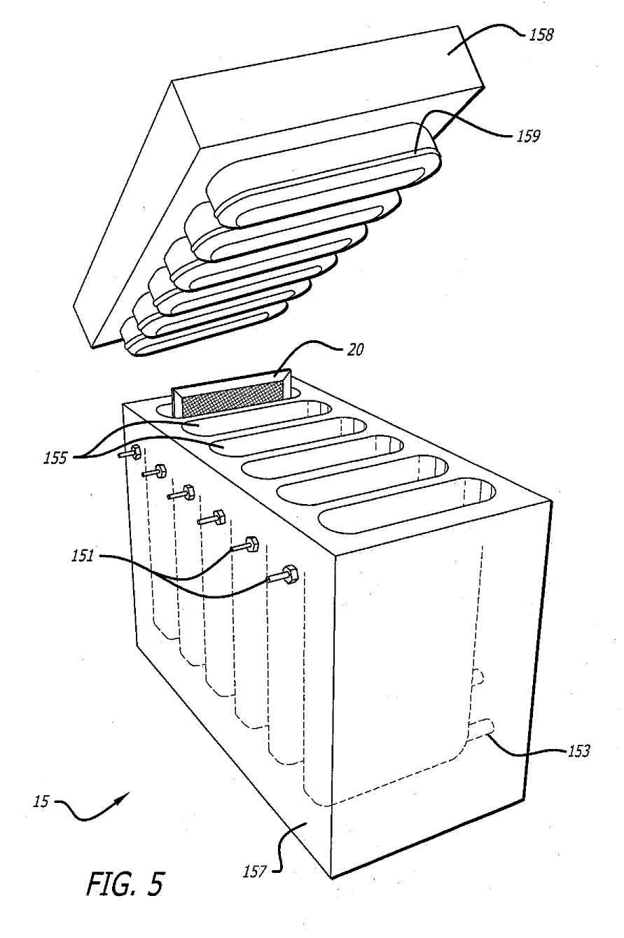

Turning attention now to FIG. 4, there is shown an embodiment of bioreactor 15. Although many configurations are available and will be understood by a person of ordinary skill in the art, the embodiment shown in FIG. 4 comprises a plurality of neoplasmic tissue slice apparatus chambers 155 (see FIG. 5). Each tissue slice apparatus chamber 155 is formed by a sealed cavity when bioreactor base 157 and bioreactor cover 158 are interconnected. Gas valves 151 may are typically connected to gas supply 36 and are in gas communication with tissue slice apparatus chamber 155 for the purpose of providing tissue slices contained in tissue slice apparatus chamber 155 with a supply of a desired gas mixture. Culture medium valves 153 are in fluid communication with tissue slice apparatus chamber 155 and serve the same function for culture medium as gas valve 151 does for gas. According to embodiments, bioreactor cover sealers 159 seal bioreactor cover 158 and bioreactor base 157. Bioreactor cover sealer 159 may be an O-ring or other similar device that prevent fluid leakage when bioreactor base 157 and bioreactor cover 158 are in an interconnected configuration, and when inverted the actual choice of device serving as bioreactor cover sealer 159 will be understood and appreciated by a person of ordinary skill in the art.

According to an embodiment shown in FIG. 5, each neoplasmic tissue slice apparatus chamber 155 accommodates at least One tissue slice apparatus 20. When uninterconnected, tissue slice apparatus 20 may be inserted into a cavity forming a part of tissue slice apparatus chamber 155 in bioreactor base 157. According to the exemplary embodiment, a single neoplasmic tissue slice apparatus 20 is inserted into each cavity; a plurality of neoplasmic tissue slice apparatuses 20, however, may be employed in a single bioreactor 15 by providing a plurality of tissue slice apparatus chambers 155 in a bioreactor 15. Nonetheless, the present disclosure contemplates configurations of bioreactor 15 that comprise various numbers of neoplasmic tissue slice apparatus chambers 155, and various numbers of tissue slice apparatuses 20 per neoplasmic tissue slice apparatus chamber 155. After each tissue slice apparatus 20 is inserted into neoplasmic tissue slice apparatus chambers 155, bioreactor cover 158 is interconnected with bioreactor base 157. Once interconnected, bioreactor 15 is sealed with bioreactor cover sealer 159. Bioreactor 15 may then be placed into incubator 52 and connected with gas supply 36 and culture medium supply 38 and experiments accordingly conducted.

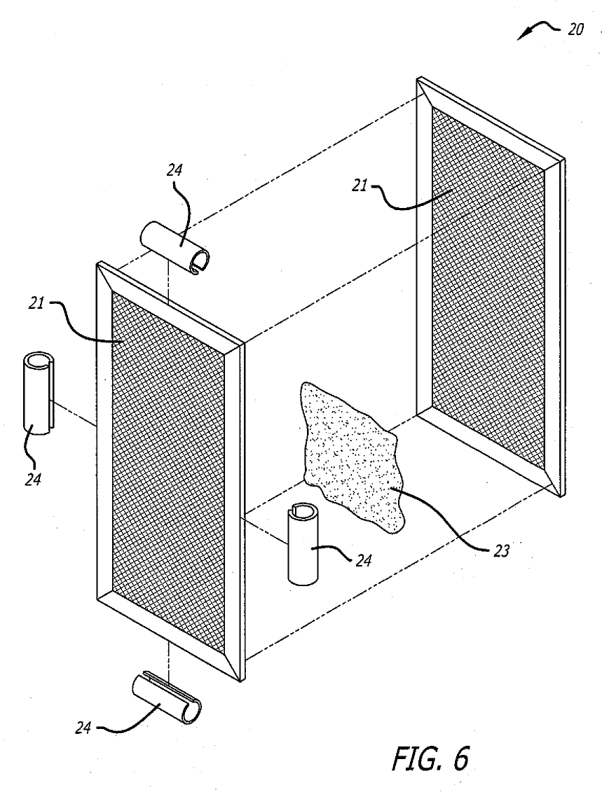

As previously described and as shown by an embodiment in FIG. 6, neoplasmic tissue slice apparatus 20 may comprise a plurality of meshes 21. Meshes 21 may be made of stainless steel or other materials that would be known by a person of ordinary skill in the art, as described previously. One or more tissue slices 23 are placed between adjacent meshes 21 and meshes 21 are clasped together with tissue slice apparatus clips 24. It will be understood by artisans that size and thickness of neoplasmic tissue slices may need to be optimized for each protocol and may vary from experiment to experiment and tissue to tissue.

According to the exemplary embodiment shown in FIG. 6, two meshes 21 form neoplasmic tissue slice apparatus 23. The tissue slices 23 are disposed in the lower 40% of neoplasmic tissue slice apparatus 20 according to the exemplary embodiment. This configuration of tissue slices 23 in tissue slice apparatus 20 ensures that the tissue slice is fully exposed to both the dry and wet cycles.

FIGS. 7A and 7B show similar embodiments of neoplasmic tissue slice apparatus 20. Two stainless steel meshes 21, the size of which can be chosen based on the dimensions of the chamber, are included. These two meshes are preferably arranged in parallel. In an embodiment, the meshes have about a 0.26 mm pore size. Also, in an embodiment, the meshes are pressed to ensure consistent flatness. Between meshes 21 is a plurality of tissue slices 23, such as liver slices arranged in an orderly fashion. The two meshes are positioned on each side of the neoplasmic tissue slices with enough room so as to not crush the neoplasmic tissue slices, but also to hold them sufficiently so that they do not get washed away by the culture medium. Although FIGS. 7A and 7B show a relatively small number of tissue slices positioned between the meshes, it is to be understood that the efficiency of the apparatus is dependent upon the number of tissue slices and sizes of the tissue slices employed. Additionally, although two meshes 21 are shown, it is contemplated that any number of meshes 21 may be used. If a single mesh 21 is used, it is formed to surround, at least partially, neoplasmic tissue slices 23 thereby forming a space and to retain them in that space. For example, the mesh could be formed in a suitably dimensioned U-shape.

Neoplasmic tissue slices 23 used in the present disclosure may be obtained from a suitable source, depending on the intended use of the apparatus. Neoplasmic tissues are expressly contemplated and may be used interchangeably with animal tissues. However, those skilled realize inherent benefits of neoplasmic tissue. Tissue slices 23 may be of any size or shape suitable for maintaining the viability and essential functions thereof. In the present disclosure thickness of tissue slices 23 shown to be effective have a thickness ranging from about 10 μm to about 2,000 μm. A thickness from about 100 μm to about 500 μm has been determined to be effective in particular experiments.

The present disclosure is ideally suited to methods of testing the toxicity and efficiency of a drug. The testing is accomplished by exposing tissue slices to a drug or drug candidate and observing the ability of the tissues, such as liver, to metabolize a compound, which compound or its metabolites can be detected. For example, ammonia and lidocaine are common compounds that can be metabolized by healthy liver. The following examples show this testing, as applied to liver-slices. Those skilled in the art will recognize the utility of the present disclosure as applied to other organs.

In order to test chemotherapeutic regimens on the tissue slices or aliquots, at least one compound is applied to at least one tissue aliquot in the bioartificial organ system. After a predetermined time elapses, data is gathered. In each experiment, the conditions may be duplicated.

Once the testing on each tissue slice or aliquot is completed, the data are compared. Comparison of the data provides for various utilities of the present disclosure, including, for example, detection of mitotic activity, cyto-toxicity parameters, and histopathology. Once derived from the data, these results are useful in formulation of the most effective chemotherapeutic regimen for a patient, for general prediction of the toxicity of a given compound or compounds, or for study of carcinogenesis, for example. Naturally, other inferences may be derived from the data, and variations in the design of the experiments using the tissue slices or aliquots allow for variations in the information derived.

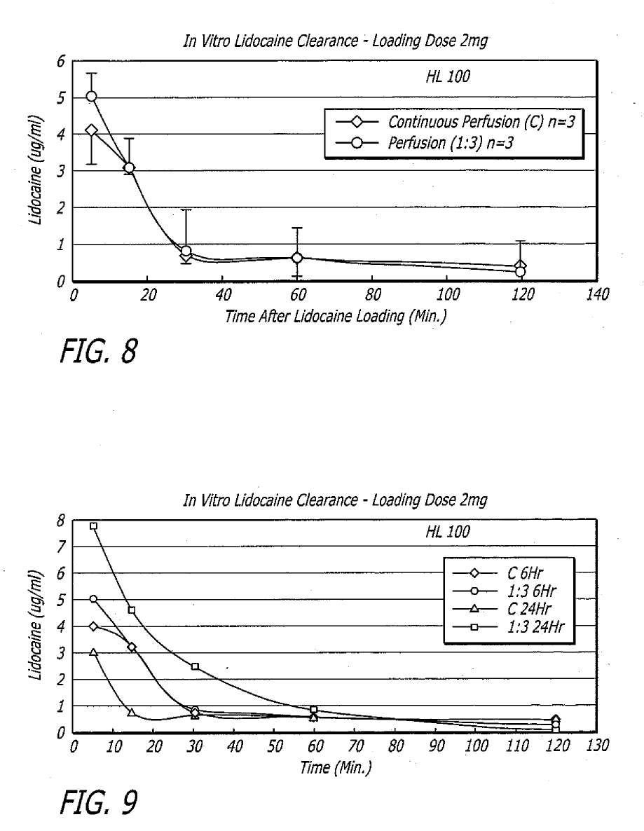

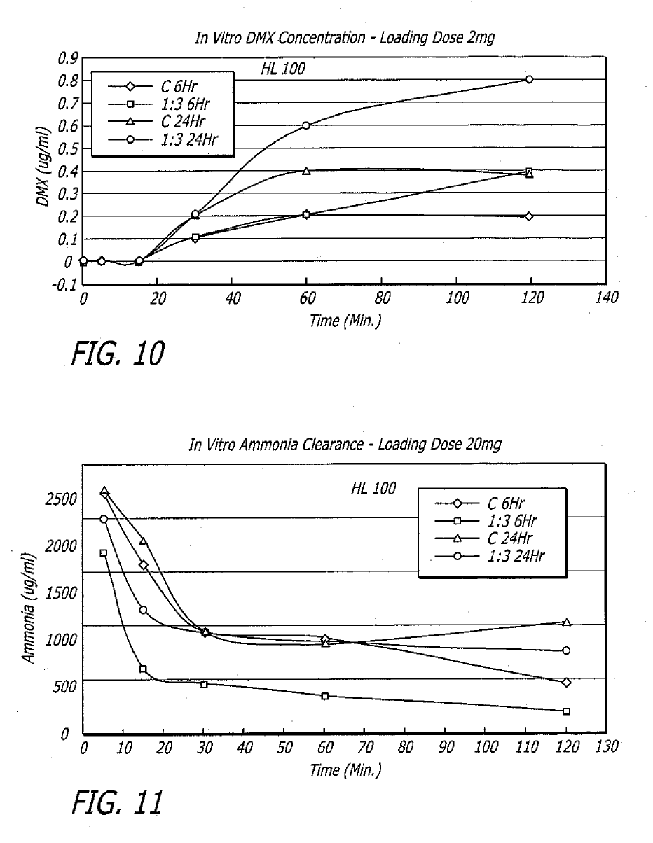

FIGS. 8-11 demonstrate the utility of the principles and apparatuses disclosed herein using liver slices as the tissue sample. The data presented in FIGS. 8 and 9 demonstrates the ability of the bioartificial organ system to clear lidocaine over time. Similarly, FIG. 10 shows the increase in concentration being managed over time, which substantially simulates in vivo physiology of samples. Finally, FIG. 11 demonstrates the ability of the teachings of the present disclosure to detoxify ammonia. The data presented in FIGS. 8-11 are not intended to be limiting or to demonstrate the actual results the teachings of the present disclosure will achieve, but merely to demonstrate the achieved utility of the teachings of the present disclosure. It is intended that various configurations will accomplish similar results from configuration to configuration that are not exactly duplicative of the data presented herein.

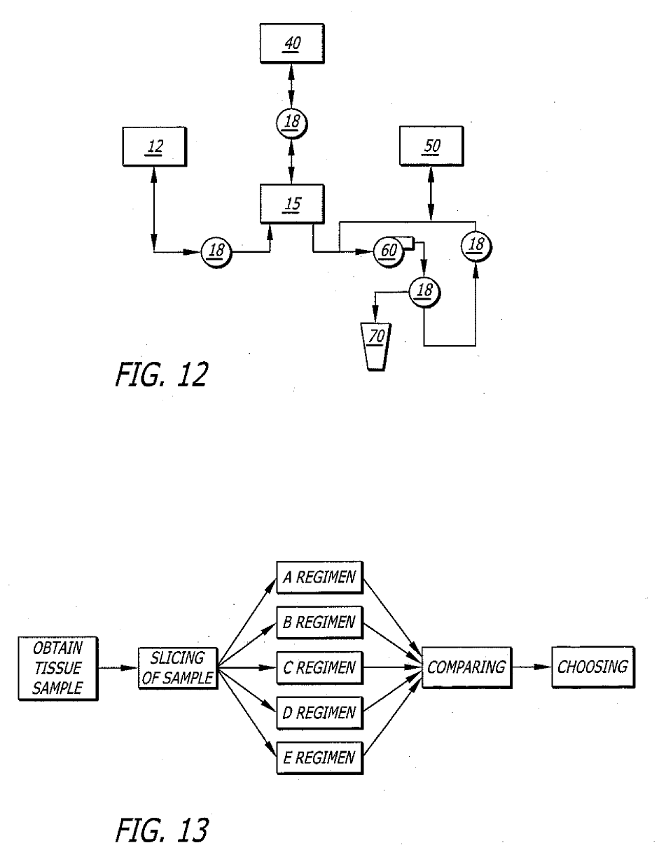

Turning now to FIG. 12, there is shown an embodiment of a method for use of the apparatuses disclosed herein. According to an embodiment, bioreactor 15 is loaded with neoplasmic tissue slices 23 and sealed. Bioreactor 15 may be identical to embodiments disclosed herein or other apparatuses with similar functionality. Bioreactor is connected to at least one culture medium reservoir 12 which contains a supply of culture medium. According to embodiments in which bioreactor comprises a plurality of tissue slice apparatus chambers 155, different culture medium reservoirs may be used to supply culture medium depending on the specific goals of the experiment sought. For example, according to an embodiment, bioreactor 15 comprises 6 tissue slice apparatus chambers 155 (see, e.g., FIG. 5). The first chamber may be used as a control wherein culture medium is supplied with no additives. The other 5 chambers may be supplied with culture medium containing a compound to be tested on the tissue slices 23 such as lidocaine or ammonia in various concentrations. Similarly, all or some of tissue slice apparatus chambers 155 may be supplied with the exact same compounds in order to have multiple sets of results or to prevent fouling of a particular tissue slice apparatus chamber 155 from preventing retrieval of results. The exact experimental design and protocol, however, are configurable in many variations as would be known and understood by a person of ordinary skill in the art. According to embodiments, filter 18 may be disposed between culture medium reservoir 12 and bioreactor 15.

Bioreactor 15 is also connected to a gas supply 40. Gas supply may supply gasses in various combinations and concentrations according to experimental designs and protocols. Typically, a single gas supply 40 may be connected to all tissue slice apparatus chambers 155. Nevertheless, a plurality of gas supplies 40 may be used if desired and called for by experimental design or protocol. According to embodiments, filter 18 may be disposed between gas supply 40 and bioreactor 15.

After culture medium and gas is supplied to each tissue slice apparatus chamber 155 bioreactor 15 is incubated for a given period of time. During incubation, tissue slices are alternately exposed to culture medium and gas. This may be accomplished in multiple ways. For example, culture medium and gas may be injected and recovered alternately so that either gas or culture medium is in tissue slice apparatus chamber 155 at any one given time. Alternately, bioreactor 15 may be rotated so that tissue samples are alternately exposed to culture medium and gas, which are both held in tissue slice apparatus chamber 155. This may be accomplished by ensuring that tissue samples 23 occupy only a certain volume of tissue slice apparatus chamber 155 so that it is fully submerged in culture medium in one configuration, but upon rotation is fully exposed to gas. FIG. 6 demonstrates an embodiment reflecting this idea, wherein tissue sample 23 occupies 40% of tissue slice apparatus 20. Other variations on this idea will be understood by a person of ordinary skill in the art.

At various points during an experiment, samples of culture medium may be removed for testing. According to the embodiment shown in FIG. 12, drain pump 60 may remove an aliquot of culture medium. Once removed, culture medium may have any gas extracted at the same time captured in bubble trap 70. Thereafter, the culture medium aliquot is held and tested in a processed culture medium reservoir 50. Once tested, it may be returned to bioreactor 15 or discarded. Filters 18 disposed within the system maintain sterility.

An embodiment shown in FIG. 13 illustrates a method of testing neoplasmic tissues using various compounds and substances. In the exemplary method, a tissue sample is extracted during surgery. Tissue is preferred to be neoplasmic. For example, to create a personalized chemotherapeutic regimen, tissue is removed from the patient for whom the regimen is to be created. For example, cancerous tissue may be removed and exposed to a variety of cancer fighting drug cocktails to determine the best cocktail for the particular cancer tested. Similarly, in toxicity testing applications, tissue from any suitable neoplasmic or animal host may be used. According to embodiments, animal tissue may be used initially. After a compound is deemed safe in animal studies, neoplasmic tissue sample may then used to further test toxicity of the compound or substance.

Tissue slices may be obtained incidental to other surgeries or in procedures designed specifically to obtain the tissue sample, for example a biopsy. For personalized chemotherapeutic regimens, tissue must be obtained from the patient, necessitating taking a tissue sample directly from the patient either during a non-related surgery or during an operation specifically designed to obtain a tissue sample. Other chemosensitivity applications may use other sources of tissue samples depending on the particular protocol.

According to embodiments, adequately sized tissue samples are used to obtain results when various compounds are tested on them. Such results, including personalized medicine related matters, are within the normal skill level of artisans and likewise may be found in at least one of U.S. Pat. Nos. 6,678,669; 6,905,816; 6,983,227 and 6,999,607 each of which are expressly incorporated herein by reference as if fully set forth herein.

Once removed from the patient, the tissue sample is sliced or otherwise divided into at least one aliquot of tissue. In an embodiment, each slice or aliquot is then individually cultured in tissue slice apparatus chamber 155 and bioreactor 15 as previously described, such as using the method shown in FIG. 12. The use of multiple, duplicative tissue slices allows researchers and doctors to expose the same tissue slices to compounds at the organ level where the only variable at play is the regimen administered.

Thereafter, the results are analyzed. Because the only variable is the regimen administered, the present disclosure provides a powerful tool for evaluation the efficacy of each given regimen compared to other viable regimens. Moreover, because the present system and methods are designed to allow testing at the organ system, and in some cases, organism level, as described in the examples below. Thus, using the methods and apparatus of the present disclosure, researchers and doctors have a powerful tool to evaluate mitotic activity, cyto-toxicity parameters, histopathology, and many other related applications at the organ, system, and organism level.

According to an embodiment, a system or organism can be recreated in vitro using the instant techniques, but provide results that are indicative of in vivo processes by using tissue slices from a variety of tissues in a system and connecting them in parallel. Connection occurs via transfer of culture medium from one tissue type to the next, which allows researchers to observe the stepwise effects of a regimen on various organ samples.

According to similar embodiments, tissue slices or different tissue types from tissue slices may be combined in single tissue slice apparatus 20 in various permutations. Experiments of this type would allow researchers to control for tissue type in an in vivo system in an in vitro environment for study and therapeutic applications. The many variations in the use of the apparatuses and methods of the present disclosure in the observation of the effects and efficacy of regimens will be understood by a person of ordinary skill in the art. Similarly, the various methods of controlling variables in an in vivo system will appeal to artisans as a powerful means of obtaining results that would otherwise be impossible short of neoplasmic or animal experiments.

For example, according to an embodiment, a plurality of neoplasmic liver slices are positioned securely within bioreactor 15 so as to maximize the surface area of the liver slices exposed to a culture medium. There is a means for selectively supplying and removing culture medium to the tissue slice apparatus chamber so that the culture medium in the chamber rises to come into contact with the tissue slices. The culture medium rises in the chamber so that the liver slices are completely immersed. The same process reversed may also be used to remove the culture medium from contact with the tissue slices. There is also a means for supplying a gas to the top of the chamber so that the tissue slices are exposed alternately to the gas and to the culture medium. This is done as described previously. Additionally, a reservoir is provided for containing the culture medium as it enters and exits the chamber. The chamber is preferably thermoregulated. For neoplasmic tissue slices, the temperature is preferably kept at about 36.5 degrees C. For rodent tissue slices, it is kept between about 36 to 38 degrees C. However, pig tissue slices are very sensitive to temperature fluctuation and it must be maintained at 38 degrees C., the normal body temperature of pigs.

### EXAMPLE 1

In an embodiment, a doctor may use the teachings of the present disclosure to determine the optimum concentrations of a chemotherapy drug cocktail for a cancer patient. The doctor takes a biopsy of a cancerous tissue from the patient and divides the tissue sample into aliquots. The doctor divides aliquots into two groups. The doctor uses the first group to determine the most effective drug cocktail for the patient in question. The doctor then uses the second group of aliquots to determine the optimal concentration of the drug cocktail.

With the first group of aliquots, the doctor uses the group to determine the most effective drug cocktail. Tissue aliquots are cultured as described previously. Various drug cocktails are administered to the tissue aliquot. The percent of apoptosis of the cancerous tissue in the aliquots is measured by methods that would be common to a person of ordinary skill in the art. The doctor then selects the drug cocktail inducing the greatest degree of apoptosis in cancerous tissue compared to the healthy tissue.

The doctor then uses the second group of tissue aliquots to determine the optimal concentration of the drug cocktail to use on the cancerous tissues. Tissue aliquots are cultured in the same manner as the first group of tissue aliquots. The doctor applies various concentrations of the drug cocktail to each tissue aliquot and selects the concentration of the drug cocktail that imparts the greatest degree of apoptosis in cancerous tissue as compared to healthy tissue.

As a result of optimizing the chemotherapeutic regimen that the patient will receive to treat the cancer, the patient will receive the treatment that imparts the greatest benefit while minimizing undesirable side effects. If the doctor wishes, the same result may be obtained in a single experiment where various concentrations of a plurality of drug cocktails is applied to a plurality of tissue aliquots.

### EXAMPLE 2

In another embodiment, the teachings of the present disclosure may be used to predict the toxicity of compounds to healthy tissue. Such results would be useful in data generated and submitted to the FDA pursuant to approval of a new drug application or abbreviated new drug application. An animal or neoplasmic tissue sample is obtained by taking a biopsy or as part of surgery. Usually two species of animals, one rodent and one non-rodent are used because a drug may affect one species differently than another. Other organs likewise provide key data and are useful within the scope of the present disclosure. In another embodiment, the tissue is taken as part from a deceased organ donor, cloned, regenerated or otherwise supplied by techniques known to those skilled in the art ranging from cord blood to stem cells by somatic cell transfer, among other things.

Tissue aliquots are derived from the tissue sample and cultured as previously described. Once the tissue is cultured, a compound is applied to each tissue aliquot to determine the efficacy of the compound to achieve a desired result as described previously. Data is gathered and interpreted as prescribed by the FDA or as according to parameters set by a person of ordinary skill in the art in the determination of a compound's efficacy in a tissue.

### EXAMPLE 3

Similarly, disease studies may be performed by using various compounds in the study of a disease. For example, inhibitors and stimulators of compounds in the tissues may be used to study their effects on chemical pathways in the tissue. Moreover, compounds may be applied to the tissue in an effort to observe their effects on the tissue level as opposed to the cellular level or organism level. The present disclosure provides the methods to use a bioartificial organ system; experimental parameters would be readily apparent to a person of ordinary skill in the art without the need for undue experimentation.

The testing may be performed by an independent third party in order to rule out any appearance of bias. Every effort is made to ensure that as few animals as possible are used as a source of tissue samples, and that they are treated neoplasmicely. Alternately, the present disclosure also contemplates the use of neoplasmic tissues, samples of which may be obtained from organ donors. Since most drugs are metabolized in the liver, toxicity studies naturally focus on the effects on the liver.

### EXAMPLE 4

The present disclosure also provides a novel way to observe the interaction between organ systems. Tissue slices may be obtained from a variety of organs. These then are placed in parallel into multiple bioreactor tissue slice apparatus chambers. Culture medium is applied to a first chamber and permitted to come into contact with the tissue slice for a time period. Following the initial time period, the culture medium is removed from the first tissue slice apparatus chamber and moved into a second tissue slice apparatus chamber containing a tissue sample from a different organ or the same organ under different experimental conditions, such as increased metabolism, a different primary cell type, or a different concentration of cell type of interest. Prior to or concurrently with moving the culture medium to the second tissue slice apparatus chamber, samples of the culture medium may be obtained to interim testing, in embodiments. The culture medium moved into the second tissue slice apparatus chamber is then reacted for a time period. The procedure is repeated for each tissue slice apparatus chamber until the experiment is concluded.

For example, a researcher may be interested in protein digestion. Samples of tissue may therefore be taken from inside the mouth, esophagus, stomach, small intestine sections corresponding to the duodenum, jejunum, and ileum, and the large intestine. A sample of culture medium with a protein sample may be therefore reacted with each disparate tissue type to determine the effect of a particular organ on the proteins to be digested on a system level.

While the apparatus and method have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A method for using neoplasmic tissue for drug testing and screening comprising:
providing a bioreactor for substantially simulating in vivo tissue function in vitro;
providing for the bioreactor to hold at least one aliquot of a tissue sample; and
allowing at least one aliquot to generate useful data.

2. The method of claim 1, wherein in vivo conditions are accomplished by maintaining cell-to-cell interaction in the tissue.

3. The method of claim 1, wherein neoplasmic tissue based data is generated by using the combination of the tissue sample and bioreactor.

4. A The method of claim 1, wherein the bioreactor is used to at least one of: determine an optimal chemotherapeutic regimen, predict toxicity of a regimen, or study disease.

5. The method of claim 1, wherein the data describes at least one of the group mitotic activity, toxicity studies, and histopathology.

6. The method of claim 1, wherein the bioreactor substantially duplicates tissue function at an organism level.

7. The method of claim 6, wherein the bioreactor substantially duplicates tissue function at a system level.

8. The method of claim 7, wherein the bioreactor substantially duplicates tissue function at an organ level.

9. The method of claim 1, wherein the useful data is generated by the application of at least one regimen.

10. A business method for neoplasmic tissue testing which comprises providing a bioreactor-based system for housing neoplasmic tissue slices;
populating the bio-reaction based system with neoplasmic tissue;
testing a regimen on the tissue; and
collecting results.
